(19) United States
(12) Patent Application Publication
(10) Pub. No.: US 2013/0274590 A1
(43) Pub. Date: Oct. 17, 2013

(54) METHOD AND APPARATUS FOR GENERATING A SIGNAL INDICATIVE OF MOTION OF A SUBJECT IN A MAGNETIC RESONANCE APPARATUS

(71) Applicants: Vincent Auboiroux, Ambilly (FR); Magalie Viallon, Lyon (FR); Lorena Petrusca, Villeurbanne (FR); Rares Salomir, Ambilly (FR)

(72) Inventors: Vincent Auboiroux, Ambilly (FR); Magalie Viallon, Lyon (FR); Lorena Petrusca, Villeurbanne (FR); Rares Salomir, Ambilly (FR)

(21) Appl. No.: 13/448,630

(22) Filed: Apr. 17, 2012

(51) Int. Cl. A61B 5/055

(52) U.S. Cl. 600/411

(57) ABSTRACT

In a method and magnetic resonance (MR) apparatus for implementing an MR-guided procedure, an MR-compatible digital camera is placed in the patient receiving opening of the MR data acquisition unit that is operated to acquire MR data for reconstructing images that are used to guide the MR-guided intervention. The digital camera is operated to obtain digital images of the exterior of the patient, from which motion of the patient is detectable. The images are analyzed in a processor to identify therefrom the motion of the patient and the result of the analysis is represented as a processor output that is used to control the timing, with respect to the motion of the examination subject, of the occurrence of at least one event in the MR-guided procedure. One important application is respiratory gating/triggering of HIFU sonication for the treatment of moving organs.

### BACKGROUND OF THE INVENTION

1. Field of the Invention

The present invention concerns the generation of a signal indicative of motion of a subject in a magnetic resonance (MR) apparatus, and in particular to the generation of such a signal for triggering an event in an MR-guided procedure.

2. Description of the Prior Art

Many types of procedures are implemented by interaction with an examination subject while the examination subject is located in a magnetic resonance apparatus, namely while the subject is located inside the data acquisition unit of such a magnetic resonance apparatus. One example of such a procedure is treatment with high intensity focused ultrasound (HIFU).

The principle of HIFU treatment is to concentrate a high acoustic intensity within a focal spot having a size of a few millimeters, in order to produce sharply localized mechanical or thermal effects. This treatment is implemented using an external source of energy, and a propagating ultrasound beam. When such a treatment is implemented to apply HIFU to a focal spot located in moving tissue, such as movement caused by respiration, such movement or motion of the organ in which the focal spot is located must be taken into account when dynamically steering the HIFU beam.

As described by Fischer et al. in “Focused Ultrasound as a Local Therapy for Liver Cancer,” Cancer Journal, Vol. 16, No. 2 (2010) pgs. 118-124, the primary challenges to HIFU therapy in the abdomen are to manage the complex motion of abdominal organs, and to prevent or avoid the risk of collateral heating at bone interfaces.

If the tissue motion management during HIFU sonication is not accurate, this may result in under-treatment of the target tissue, and/or unwanted collateral damage to healthy or critical surrounding anatomical structures.

It is known to guide a HIFU treatment procedure based on acquired MR images, which means that the HIFU therapy must then be administered to the subject while the subject is located in the examination volume of an MR data acquisition unit of the MR apparatus. In general, such MR-guided HIFU therapy requires motion encoding or motion monitoring using a detection device that is compatible with the radio-frequency fields and magnetic fields that are generated in the examination volume of such an MR data acquisition unit, and real time processing of motion information and feedback to the beam-steering system. In principle, this motion monitoring can be achieved by analyzing the magnetic resonance images themselves, but this requires compromises with respect to certain parameters (temporal resolution v. signal-to-noise ratio (SNR)), or image contrast.

One straightforward approach in this context is the use of respiratory gating. This means that HIFU sonication takes place periodically, during each quite or rest phase of the respiratory cycle, i.e., during exhalation. Respiratory gating generally increases the treatment time. Conventionally, some type of belt or respiratory cushion has been used to detect and monitor the respiration curve, or a volume and/or pressure signal from a mechanical ventilator can be used to determine the on/off sonication periods, with the patient being under general anesthesia.

Another known approach is to generate an atlas of motion fields during an initial learning phase of a control unit, based on magnitude data of temperature-sensitive GRE acquisition. This procedure is disclosed by deSenneville et al., in “Real-time Adaptive Methods for Treatment of Mobile Organs by MRI-Controlled High-Intensity Focused Ultrasound,” Magnetic Resonance in Medicine, Vol. 57, No. 2 (2007) pgs. 319-330. In this approach, the motion field of the most similar image in the atlas is used to correct the target position. Under the hypothesis of periodic motion, the focal point position for the next cycle is then estimated. This procedure, however, can only manage liver deformations caused by the periodic breathing cycle, and is not capable of handling the non-rigid liver deformations that are caused by intestinal activity or muscle relaxation, as noted by von Siebenthal et al. in “4D MR Imaging of Respiratory Organ Motion and its Variability,” Phys. Med. Biol., Vol. 52, No. 6 (2007) pgs. 1547-1564. In general, T2*-weighted MR magnitude data from a gradient echo sequence dedicated to fast MR thermometry generally lacks anatomical contrast. Another approach is to make use of a pencil-beam navigator, as described by Hardy et al. in “Rapid NMR Cardiography with a Half-Echo Mode-Method,” Journal of Computer Assisted Tomography, Vol. 15, No. 5 (1991) pgs. 868-874, and this could be used for motion compensation of thermometry imaging, and to provide information about the target motion to the HIFU system for focal spot adjustment. MR information-based real-time motion compensation, however, generally comprises spatial resolution, geometric distortion, and the precision of the MR thermometry, as reported by Ries et al., “Real-time 3D Target Tracking in MRI Guided Focused Ultrasound Ablations in Moving Tissues,” Magnetic Resonance in Medicine, Vol. 64, No. 6 (2010) pgs. 1704-1712.

A first attempt at ultrasound-based motion tracking during MR-guided HIFU was reported in phantoms undergoing periodic and rigid motion of a small amplitude, deOliveira et al., “Rapid Motion Correction in MR-Guided High-Intensity Focused Ultrasound Heating Using Realtime Ultrasound Echo Information,” NMR Biomed., Vol. 23, No. 9 (2010) pgs. 1103-1108. Continuous one-dimensional ultrasound echo detection along a direction parallel to the main axis of motion was used. This setup is not suitable for clinical application, because the external ultrasound imaging probe cannot emit ultrasound parallel to the axis of respiratory motion. Moreover, the local motion in liver is spatially dependent, so as one-dimensional projection would not be sufficient.

By contrast to images obtained with ultrasound, external images of the subject do not provide tomographic information from the interior of the body. External images, however, have the advantages of being acquired with a relatively simply implementation, with no need for finding an additional acoustic window as is the case for ultrasound imaging, and there is no sensitivity to the HIFU emission. An optical camera is not influenced by the ultrasonic waves, and is virtually insensitive to electromagnetic radiation from the HIFU hardware.

In the context of brain MRI, it has been recently suggested to track the patient using a camera located inside the patient-receiving opening (bore or tunnel) of the MR data acquisition unit using an in-bore camera and a checkerboard marker attached to the forehead of the patient. Such an approach is described in Forman et al., “Self-Encoded Marker for Optical Prospective Head Motion Correction in MRI,” Med. Image Anal., Vol. 15, No. 5 (2011) pgs. 708-719. This article describes the use of a self-encoded marker with each feature on the pattern being augmented with a 2D barcode, tracked by a single analog in-bore camera attached to the head MR-coil. Outside of the scanner room, the analog video signal is converted to a digital signal using a frame grabber. This technology has been used for the correction of fMRI data, but has not been used in the context of image-guided therapy. Motion correction with this approach encompassed a rotation of 18° around the principle axis of the cylindrical phantom in between two scans. After rigid registration of the resulting volumes, a maximum error of 0.39 mm and 0.15° in translation and rotation were measured, respectively.

### SUMMARY OF THE INVENTION

An object of the present invention is to provide a method for taking motion of an examination subject into account in an MR-guided intervention procedure, wherein the effective administration of the therapy is dependent on the therapy occurring at a desired point in time with respect to motion exhibited by the subject

A further object of the present invention is to provide an MR apparatus to implement such a method.

The first object is achieved in accordance with the present invention by a method for MR-guided therapy administration wherein the therapy is administered to a patient inside of a magnetic resonance data acquisition unit, the patient exhibiting extracorporeally detectable motion, such as periodic motion due to respiration. While the examination subject is located inside the MR data acquisition unit, MR image data are acquired from the subject from which MR images are reconstructed that are used to guide the administration of the therapy in terms of appropriately identifying an intracorporeal site at which the therapy is to be administered. The site is located in an organ of the patient that is subject to the aforementioned motion. In accordance with the invention, the motion is detected by placing a digital camera inside the patient-receiving opening of the MR apparatus, and obtaining digital images with the camera that are then analyzed in a computerized processor to identify information therefrom indicative of the motion. This information is then used to generate an electrical signal at an output of the processor in a form that allows the therapy to be implemented at a desired point in time with respect to the motion.

The signal that is generated that is indicative of the motion may be a continuous signal, such as a continuous signal representing respiratory movement, or may be a trigger signal that is emitted upon the detected motion exhibiting a particular characteristic, such as a spatially-dependent characteristic.

The analysis of the motion represented in the digital images can take place using a suitable pattern recognition algorithm, comparison algorithm, amplitude detection algorithm, or any other appropriate image processing algorithm that is able to detect and track motion from an analysis of the successive digital images.

The therapy procedure may be, for example, the administration of HIFU, with the on-off times of HIFU being controlled dependent on the identified motion. Another example is MR-guided Acoustic Radiation Force Imaging (ARFI) wherein the data acquisition is triggered dependent on the respiratory cycle of the patient, as indicated by the detected motion, so that data acquisition takes place when the patient is exhibiting the least amount of movement, such as at the end of exhalation.

The second object noted above is accomplished by an MR imaging apparatus designed to implement the method described above, using a digital camera that is constructed with all of its components being formed of MR-compatible material, and being located inside the patient-receiving opening of the data acquisition unit of the MR apparatus, with appropriate RF shielding surrounding the camera inside the patient-receiving opening. The digital images obtained with the camera are communicated via a shielded cable.

The camera may be provided with a high-power light emitting diode (LED) that can be appropriately operated continuously or intermittently to provide sufficient light to obtain the optical images, when the digital camera is simultaneously operated to obtain those images.

### BRIEF DESCRIPTION OF THE DRAWINGS

FIG. 1 schematically illustrates the basic components of a magnetic resonance imaging system constructed and operating in accordance with the present invention.

FIG. 2 schematically illustrates the data acquisition unit of the magnetic resonance apparatus of FIG. 1, showing the digital camera inside the patient-receiving opening thereof, in accordance with the present invention.

FIG. 3 schematically illustrates a portion of an embodiment of a shielded cable for exchanging information and operating signals with the digital camera located inside the magnetic resonance data acquisition unit.

FIG. 4 schematically illustrates implementation of MR-guided HIFU therapy in accordance with the present invention.

FIG. 5 schematically illustrates an embodiment for MR-ARFI data acquisition in accordance with the present invention.

### DESCRIPTION OF THE PREFERRED EMBODIMENTS

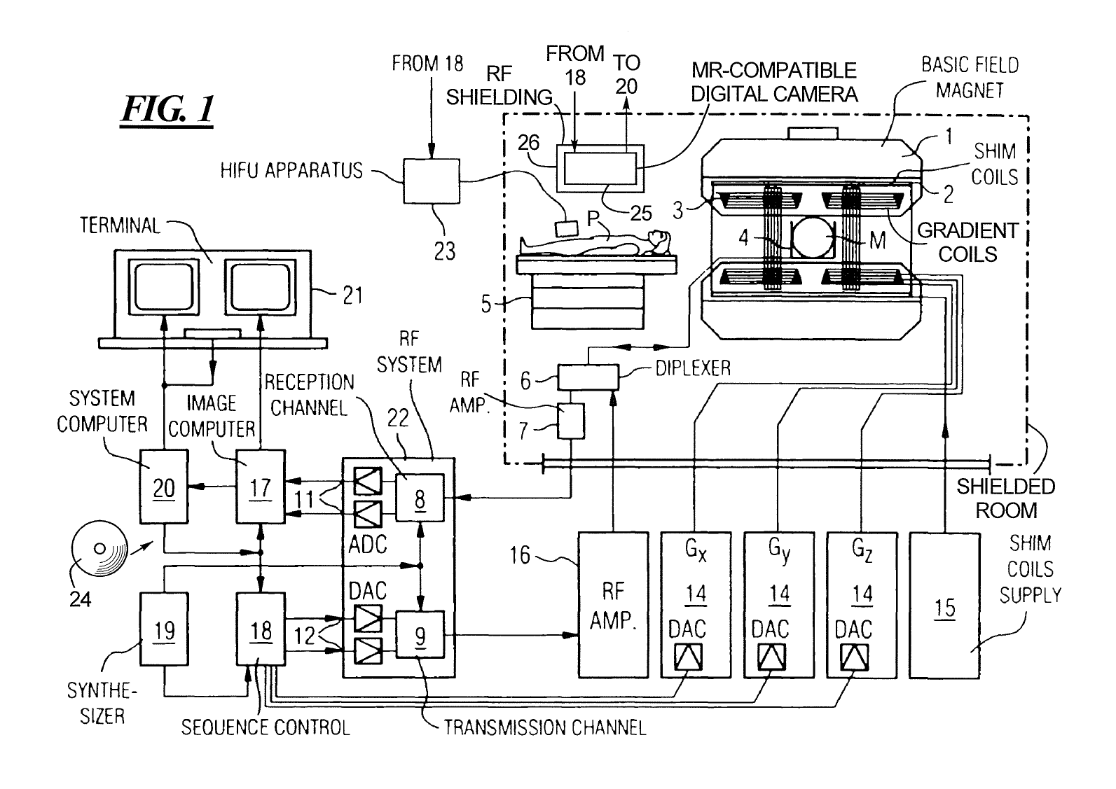

FIG. 1 is a schematic illustration of a magnetic resonance tomography apparatus operable according to the present invention. The structure of the magnetic resonance tomography apparatus corresponds to the structure of a conventional tomography apparatus, with the differences described below. A basic field magnet 1 generates a temporally constant, strong magnetic field for the polarization or alignment of the nuclear spins in the examination region of a subject such as, for example, a part of a patient P to be examined. The high homogeneity of the basic magnetic field required for the magnetic resonance measurement is defined in a spherical measurement volume M into which the parts of the patient P to be examined are introduced. For satisfying the homogeneity requirements and, in particular, for eliminating time-invariable influences, shim plates of ferromagnetic material are attached at suitable locations. Time-variable influences are eliminated by shim coils 2 that are driven by a shim power supply 15.

A cylindrical gradient coil system 3 that is composed of three sub-windings is introduced into the basic field magnet 1. Each sub-winding is supplied with current by an amplifier 14 for generating a linear gradient field in the respective direction of the Cartesian coordinate system. The first sub-winding of the gradient field system generates a gradient Gₓ in the x-direction, the second sub-winding generates a gradient G_{y} in the y-direction and the third sub-winding generates a gradient G_{z} in the x-direction. Each amplifier 14 has a digital-to-analog converter that is driven by a sequence controller 18 for the temporally correct generation of gradient pulses.

A radio frequency antenna 4 is situated within the gradient field system 3. This antenna 4 converts the radio frequency pulse output by a radio frequency power amplifier 30 into a magnetic alternating field for exciting the nuclei and alignment of the nuclear spins of the examination subject or of the region of the subject to be examined. The antenna 4 is schematically indicated in FIG. 1. For acquiring magnetic resonance data according to a PPA technique, the antenna 4 is a coil array formed by multiple individual reception coils. The antenna 4 can include a different coil for emitting the RF signals into the subject.

The radio frequency antenna 4 and the gradient coil system 3 are operated in a pulse sequence composed of one or more radio frequency pulses and one or more gradient pulses. The radio frequency antenna 4 converts the alternating field emanating from the precessing nuclear spins, i.e. the nuclear spin echo signals, into a voltage that is supplied via an amplifier 7 to a radio frequency reception channel 8 of a radio frequency system 22. The radio frequency system 22 also has a transmission channel 9 in which the radio frequency pulses for exciting the nuclear magnetic resonance are generated. The respective radio frequency pulses are digitally represented as a sequence of complex numbers in the sequence controller 18 on the basis of a pulse sequence prescribed by the system computer 20. As a real part and an imaginary part, this number sequence is supplied via an input 12 to a digital-to-analog converter in the radio frequency system 22 and from the latter to a transmission channel 9. In the transmission channel 9, the pulse sequences are modulated onto a high-frequency carrier signal having a base frequency corresponding to the resonant frequency of the nuclear spins in the measurement volume.

The switching from transmission mode to reception mode ensues via a transmission-reception diplexer 6. The radio frequency antenna 4 emits the radio frequency pulses for exciting the nuclear spins into the measurement volume M and samples resulting echo signals. The correspondingly acquired nuclear magnetic resonance signals are phase-sensitively demodulated in the reception channel 8 of the radio frequency system 22 and converted via respective analog-to-digital converters into a real part and an imaginary part of the measured signal. An image computer 17 reconstructs an image from the measured data acquired in this way. The management of the measured data, of the image data and of the control programs ensues via the system computer 20. On the basis of control programs, the sequence controller 18 controls the generation of the desired pulse sequences and the corresponding sampling of k-space. In particular, the sequence controller 18 controls the temporally correct switching of the gradients, the emission of the radio frequency pulses with defined phase and amplitude as well as the reception of the magnetic resonance signals. The time base (clock) for the radio frequency system 22 and the sequence controller 18 is made available by a synthesizer 19. The selection of corresponding control programs for generating a magnetic resonance image as well as the presentation of the generated magnetic resonance image ensue via a terminal 21 that has a keyboard as well as one or more picture screens.

The apparatus shown in FIG. 1 operates in accordance with the present invention by virtue of an appropriate pulse sequence (protocol) being entered by an operator via the terminal 22 into the system computer 20 and the sequence control 18, and programming instructions for implementing the method according to the invention that are encoded on a non-transitory data storage medium 24 that is individually loaded into the computerized system represented at least by the system computer 20 and the sequence control 18. The programming instructions may be appropriately distributed among those different units.

As also schematically indicated in FIG. 1, the apparatus includes a high intensity focused ultrasound (HIFU) apparatus 23. The HIFU apparatus 23 can be operated (activated) by a signal from the sequence control 18, which may be initiated, for example, by a signal from the system computer 20.

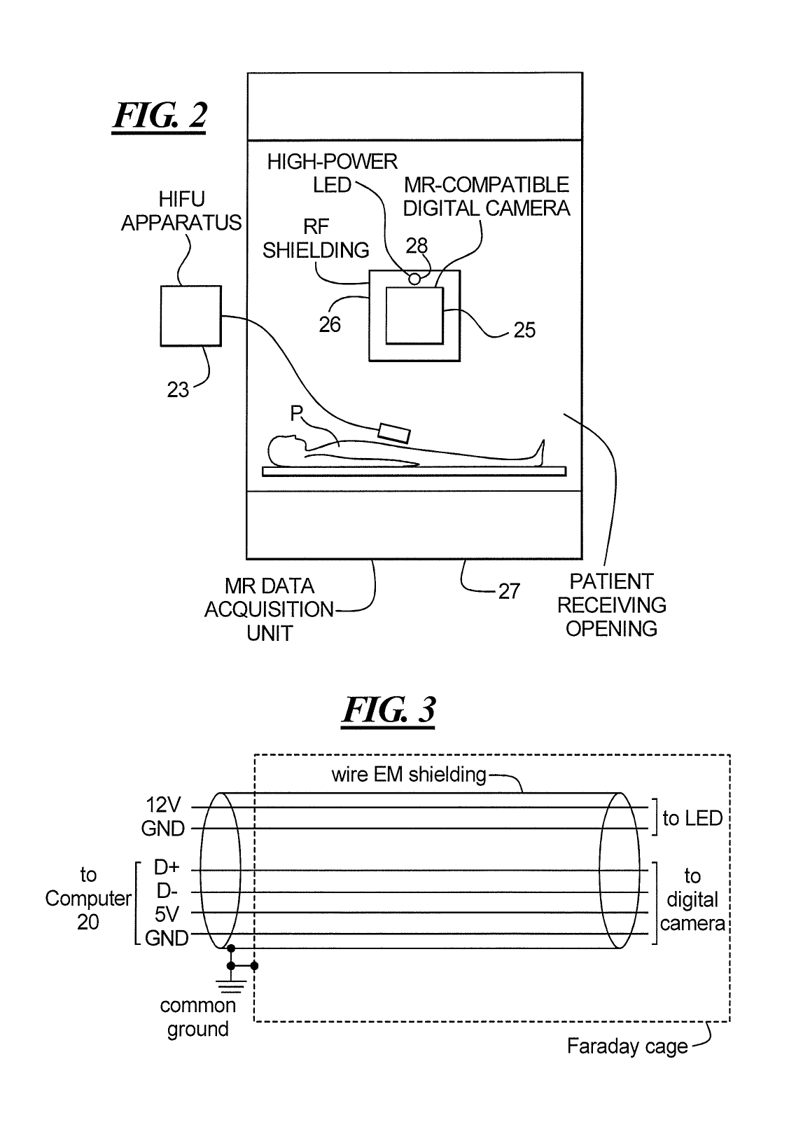

Although FIG. 1 schematically shows the MR-compatible digital camera generally located within the shielded room of the magnetic resonance apparatus, FIG. 2 shows that the MR-compatible digital camera 25 will actually be located in the patient receiving opening of the MR data acquisition unit 27. In FIG. 2, the MR data acquisition unit 27 is shown as the type of unit that has a cylindrical bore or tunnel through which the patient P is moved. It will be understood, however, that the invention can also be used in MR systems of the type known as “open magnet” systems, wherein the basic field magnet is formed by two pole pieces connected by a yolk in C-shaped arrangement.

As also indicated in FIG. 2, within the RF shielding 26 for the MR-compatible digital camera 25, there may also be included a high-power light emitting diode (LED) 28, that can be operated simultaneously with the acquisition of images by the digital camera 25, in order to appropriately illuminate the field of view of the digital camera 25.

The MR-compatible digital camera 25 can be a consumer grade USB digital camera that has been made MR-compatible by removing any magnetic parts and adding the RF shielding 26.

As schematically shown in FIG. 3, the RF shielding 26 effectively forms a Faraday cage, and the digital camera 25 can be supplied by an electromagnetically shielded cable, as shown in FIG. 3. The conductors in the shielded cable shown in FIG. 3 can include, for example, a supply line (in this example, at 12V), and a return line to ground, for operating the LED 28. The shielded cable can also include conductors D+ and D− for transferring USB data, a dc ground GND, and a supply for the USB level, in this example at 5V.

The cable shown in FIG. 3 is a multi-tiered shielded cable that proceeds through the Faraday cage through a waveguide and is connected to the digital camera at the shown location in FIG. 2 inside the patient receiving opening. The cable also proceeds to (for example) the system computer 20 shown in FIG. 1, although the system computer 20 is but one processing unit that is among the distributed computerized operating system shown in FIG. 1 that also includes the sequence control 18, the synthesizer 19 and the image computer 17.

As an alternative to the arrangement shown in FIG. 3, an additional and independent dc voltage supply line can be used to power the LED 28. The digital camera 25 is EM shielded by completely covering it with copper tape, except for a circular opening *(approximately 2 mm in size) needed for the optical aperture. The copper tape is connected to the shielding of the shielded cable shown in FIG. 3. A similar shielding is used for the LED 28.

The optical camera can be mounted to a non-magnetic orbital ring located in the patient receiving opening of the data acquisition unit 27, or alternatively can be connected to the HIFU platform.

Suitable triggers based on motion analysis of the images generated by the camera 25 can be implemented. Anatomical landmarks can be automatically set, or sharp edge-features in the optical region of interest and their displacement, can be tracked or followed using a calculation of the optical flow based on the iteratively Lucas-Kanade method in pyramids, as described by Lucas et al., “An Iterative Image Registration Technique with an Application in Stereo Vision,” Proceedings of the International Joint Conference on Artificial Intelligence (1981) pgs. 674-679. Implementation of the method in pyramids is described in Bouguet, “Pyramidal Implementation of the Lucas-Kanade Feature Tracker,” OpenCV Documentation, Intel Corp., Microprocessor Research Labs (1999). The optical data from the camera 25 can be processed at 30 fps online, with a 33 ms sampling time.

The output of the motion detecting algorithm can be a respiration curve that can be used to trigger a conventional DAC interface to the HIFU beam former substantially in real time, in order to dynamically adapt the HIFU beam steering. Alternatively, such a respiration curve can be supplied to the system computer 20 or the sequence control 18 of the magnetic resonance apparatus in order to trigger the acquisition of magnetic resonance data, such as with MR thermometry or MR acoustic radiation force imaging (ARFI), the latter being schematically shown in FIG. 5.

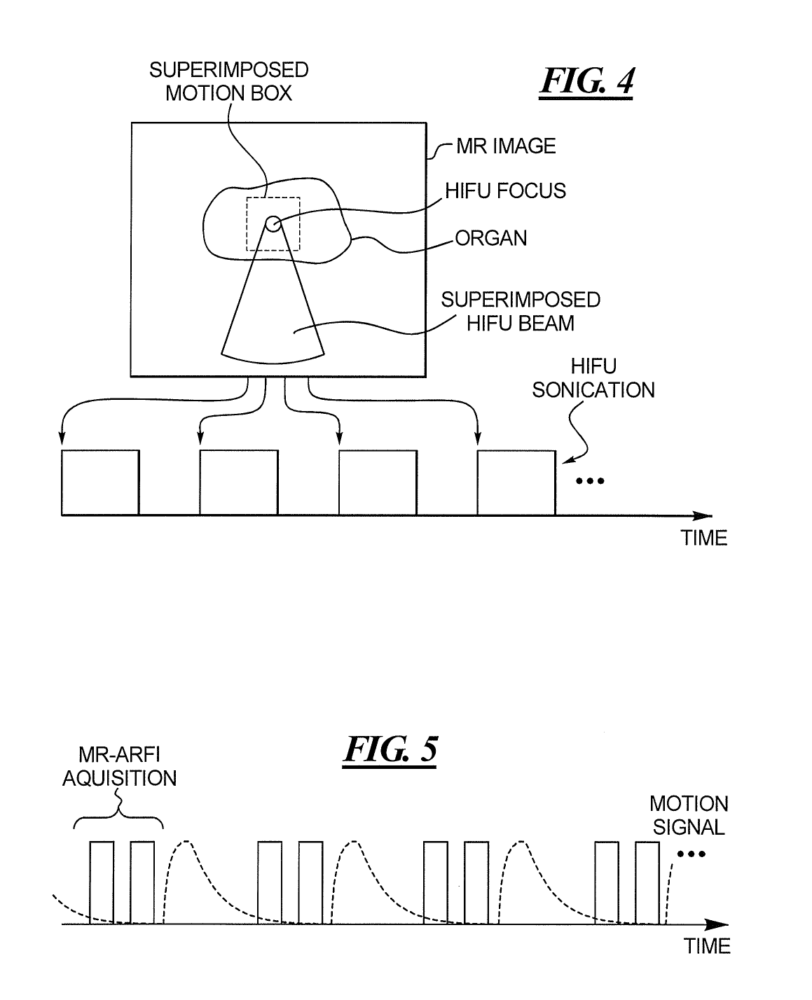

As shown in FIG. 4 the motion detection algorithm can be used to generate a motion box that can be superimposed on the acquired MR image that is conventionally used for MR-guided HIFU. The HIFU beam can also be superimposed on this image, and the HIFU focus can also be indicated. The HIFU focus is located in an organ that is to be treated with HIFU. The superimposed motion box will change in position within the MR image dependent on the motion, and the HIFU sonication can be triggered when the HIFU focus is located within the superimposed motion box. The detection algorithm can be selected or adjusted so that the motion box can be as small or large as desired, so that triggering of the HIFU sonication can occur very precisely.

Optionally, multiple cameras can be used to acquire the 3D shape of a body region, such as the abdomen, using stereoscopic reconstruction, as described in Schaerer et al., Multi-Dimensional Respiratory Motion Tracking from Markerless Optical Surface Imaging Based on Deformable Mesh Registration,” Phys. Med. Biol., vol. 57 (2012) pgs. 357-373. Correlation of respiratory motion between the external patient surface, determined from the optical data obtained with the digital camera 25 and internal anatomical landmarks, obtained from fast dynamic MRI data, may be used for prospective motion compensation during MR guided HIFU treatment. Alternatively, the external patient surface can be reconstructed using a single optical device by fringe projection profilometry, as described by Price et al., “Real-Time Optical Measurement of the Dynamic Body Surface for Use in Guided Radiotherapy,” Phys. Med. Biol, Vol. 57 (2012) pgs. 415-436.

An advantage of the invention is that the image data acquisition is contact-free, and does not place any external obstacle in the HIFU beam entry window to the treatment site. In contrast to conventional mechanical sensors, such as an abdominal belt or a pressure cushion, that are operated in a user-dependent manner and may thus complicate the abdominal interventional procedures, the inventive approach is flexible and user-independent and enables a large field of view for the motion determination.

The available frame rate and resolution of commercial digital cameras is significantly higher compared to analog standards. For example, cameras with up to 500 fps with a 1,280×1,024 pixel CMOS image sensor are available. These features are advantageous for real time motion monitoring and correction. Moreover, digital devices are inherently more robust to EM noise and perturbation, even if the aforementioned RF shielding might in some instances be sub-optimal.

By contrast to belt or cushion-type respiratory sensors, which only provide a temporal curve, in accordance with the invention 2D or 3D dynamic images can be obtained so that mapping of the surface motion is feasible, for example. Moreover, some patients may exhibit primarily thoracic breathing while others may exhibit predominantly abdominal breathing, thereby requiring appropriate and accurate location of a mechanical sensor, which is not a factor in accordance with the present invention.

Moreover, the motion detection in accordance with the present invention does not modify the patient's respiration patterns in any manner as may occur with a mechanical sensor.

The method and apparatus in accordance with the present invention can provide direct measurement of distances without a need for an indirect conversion from other parameters, such as pressure/volume or force/displacement, as is necessary with abdominal belts and pressure cushions. The calibration and response are essentially linear in accordance with the invention. The correction for any geometric distortion of the image is easily achieved with a calibration board.

The inventive method and apparatus can enable establishment of a correlation between respiratory motion and the external patient surface, and internal anatomical landmarks, as described in Fayad et al., “Technical Note: Correlation of Respiratory Motion Between External Patient Surface and Internal Anatomical Landmarks,” Med. Phys., Vol. 38, No. 6 (2011), pgs. 3157-3164.

Since the practical implementation of the invention was substantiated using a commercial-grade camera, the method and apparatus will be economically implemented in clinical practice.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for implementing a magnetic resonance (MR)-guided intervention, comprising:
placing an examination subject, exhibiting an extracorporeally detectable, substantially periodic motion, in a patient receiving opening of an MR data acquisition unit of an MR apparatus;
operating said MR data acquisition apparatus to acquire MR data from the examination subject, including generating a static magnetic field and at least one switchable gradient field and at least one radio-frequency (RF) pulse;
acquiring digital images of the exterior of the examination subject in the MR data acquisition unit with an MR-compatible digital camera in the patient receiving opening of the MR data acquisition unit;
supplying said digital images from said digital camera to a processor and, in said processor, analyzing said digital images to identify said motion of said examination subject, and generating a processor output representing said motion; and
implementing an MR-guided intervention on said examination subject guided by images reconstructed from said MR data, and controlling an occurrence of at least one event of said intervention using said processor output to cause said at least one event to occur at a selected time with respect to said motion of said examination subject.

2. A method as claimed in claim 1 comprising implementing, as said intervention, High Intensity Focused Ultrasound (HIFU) therapy, and controlling, as said at least one event, emission of a HIFU beam dependent on said processor output.

3. A method as claimed in claim 2 comprising controlling said emission of said HIFU beam by steering said HIFU beam dependent on said processor output.

4. A method as claimed in claim 3 comprising generating, as said processor output, a motion box defining a selected spatial area of the examination subject in at least one of said MR images, and steering said HIFU beam to cause a focus of said HIFU beam to be within said motion box.

5. A method as claimed in claim 1 comprising implementing, as said intervention, MR Acoustic Radiation Force Imaging (ARFI), and controlling said intervention dependent on said processor output by triggering acquisition of ARFI data dependent on said motion of said examination subject.

6. A method as claimed in claim 5 comprising generating, as said processor output, a respiration signal representing respiration of the examination subject, and triggering said acquisition of said ARFI data acquisition during an exhalation phase of said respiration.

7. A method as claimed in claim 1 comprising also placing a MR-compatible high-power light emitting diode (LED) in said patient receiving opening of said MR data acquisition unit, and operating said high power LED to illuminate a region of the examination subject encompassed by a field of view of said digital camera simultaneously with acquisition of said digital images by said digital camera.

8. A method as claimed in claim 7 comprising making said high-power LED MR compatible by enclosing said high-power LED in RF shielding on said high-power LED.

9. A method as claimed in claim 8 comprising making said digital camera MR compatible by enclosing said digital camera in a common RF shielded enclosure with said high-power LED, and supplying power to said high-power LED and to said digital camera, and transferring data representing said digital images from said digital camera, into and out of said RF shielding via an RF shielded cable.

10. A method as claimed in claim 1 comprising making said digital camera MR compatible by enclosing said digital camera in RF shielding on said digital camera and by containing no magnetic components.

11. A magnetic resonance apparatus for implementing a magnetic resonance (MR)-guided intervention, comprising:
an MR data acquisition unit having a patient receiving opening therein configured to receive an examination subject, exhibiting an extracorporeally detectable, substantially periodic motion;
a control unit configured to operate said MR data acquisition apparatus to acquire MR data from the examination subject, including generating and at least one switchable gradient field and at least one radio-frequency (RF) pulse in the presence of a multi-Tesla basic magnetic field generated by the MR data acquisition unit;
an MR-compatible digital camera in the patient receiving opening of the MR data acquisition unit configured to acquire digital images of the exterior of the examination subject in the MR data acquisition unit;
a processor supplied with said digital images from said digital camera, said processor being configured to analyze said digital images to identify said motion of said examination subject, and generating a processor output representing said motion; and
said control unit being configured to implement an MR-guided intervention on said examination subject guided by images reconstructed from said MR data, and to control an occurrence of at least one event using said processor output to cause said at least one event of said intervention to occur at a selected time with respect to said motion of said examination subject.

12. A magnetic resonance apparatus as claimed in claim 11 comprising a High Intensity Focused Ultrasound (HIFU) therapy apparatus configured to implement said MR-guided intervention, and said control unit being configured to control, as said at least one event, emission of a HIFU beam by said HIFU therapy apparatus dependent on said processor output.

13. A magnetic resonance apparatus as claimed in claim 12 wherein said control unit is configured to control said emission of said HIFU beam by steering said HIFU beam dependent on said processor output.

14. A magnetic resonance apparatus as claimed in claim 13 wherein said processor is configured to generate, as said processor output, a motion box defining a selected spatial area of the examination subject in at least one of said MR images, and wherein said control unit is configured to steer said HIFU beam to cause a focus of said HIFU beam to be within said motion box.

15. A magnetic resonance apparatus as claimed in claim 11 wherein said processor is configured to implement, as said intervention, MR Acoustic Radiation Force Imaging (ARFI), and controlling said intervention dependent on said processor output by triggering acquisition of ARFI data dependent on said motion of said examination subject.

16. A magnetic resonance apparatus as claimed in claim 15 comprising generating, as said processor output, a respiration signal representing respiration of the examination subject, and wherein said processor is configured to trigger said acquisition of said ARFI data acquisition during an exhalation phase of said respiration.

17. A magnetic resonance apparatus as claimed in claim 11 comprising also an MR-compatible high-power light emitting diode (LED) in said patient receiving opening of said MR data acquisition unit, and wherein said processor is configured to operate said high power LED to illuminate a region of the examination subject encompassed by a field of view of said digital camera simultaneously with acquisition of said digital images by said digital camera.

18. A magnetic resonance apparatus as claimed in claim 17 wherein said high-power LED is MR compatible by being enclosed in RF shielding on said LED.

19. A magnetic resonance apparatus as claimed in claim 18 wherein said digital camera is MR compatible by being enclosed in a common RF shielded enclosure with said high-power LED, and comprising and RF shielded cable that supplies power to said high-power LED and to said digital camera, and transfers data representing said digital images from said digital camera, into and out of said RF shielding.

20. A magnetic resonance apparatus as claimed in claim 11 wherein said digital camera is MR compatible by being enclosed in RF shielding on said digital camera and by containing no magnetic components.
